(19) United States
(12) Patent Application Publication
(10) Pub. No.: US 2023/0211098 A1
(43) Pub. Date: Jul. 6, 2023

(54) INFECTIOUS AEROSOL CAPTURE MASK WITH SUCTION TUBE ADAPTER FOR ACTIVE AND PASSIVE USE

(71) Applicant: Board of Regents of the University of Nebraska, Lincoln, NE (US)

(72) Inventors: Steven LISCO, OMAHA, NE (US); Nicholas MARKIN, ELKHORN, NE (US)

(73) Assignee: Board of Regents of the University of Nebraska, Lincoln, NE (US)

(21) Appl. No.: 17/999,245

(22) PCT Filed: Jun. 14, 2021
(86) PCT No.: PCT/US2021/037179

§ 371 Date: Nov. 18, 2022

Related U.S. Application Data
(60) Provisional application No. 63/038,409, filed on Jun. 12, 2020.

(51) Int. Cl. A61M 16/00; A61M 16/06; A61M 16/10; A61M 16/20; A61M 16/08; A41D 13/11

(52) U.S. Cl. CPC A61M 16/0093; A41D 13/1138; A61M 16/06; A61M 16/0009; A61M 16/208; A61M 16/0816; A61M 16/1065; A61M 2205/7509

(57) ABSTRACT

An infectious aerosol capture mask (IACM) includes a face tent coupled to a suction tube adapter. The face tent includes a proximal opening configured to be disposed over the mouth and nose of a patient. The face tent further includes a distal opening with a smaller diameter than the proximal opening. A coupler is configured to secure the suction tube adapter to the distal opening of the face tent. The suction tube adapter includes a suction port configured to be coupled to a suction tube for active capture of infectious aerosol and left unconnected for passive capture of infectious aerosol. A viral filter is disposed between the suction port and the face tent to capture infectious aerosols expelled by the patient. The IACM further includes one or more one-way valves that are configured to permit airflow into the face tent.

### CROSS-REFERENCE TO RELATED APPLICATIONS

The present application claims priority under 35 U.S.C. §119(e) to U.S. Provisional Application Serial No. 63/038,409, filed Jun. 12, 2020, and titled “Infectious Aerosol Capture Mask (IACM),” which is incorporated herein by reference in its entirety.

### TECHNICAL FIELD

The present invention generally relates to medical devices for preventing transmission of infectious diseases.

### BACKGROUND

Current standard of care does not give enough attention to protecting healthcare environments and workers from accidental exposure to aerosolized pathogens, like COVID-19. To provide safer healthcare environments, there is a need for technologies that do a better job at preventing environmental contamination and healthcare worker infection from asymptomatic or otherwise unknown infectious patients.

### SUMMARY

An infectious aerosol capture mask (IACM) is disclosed. In embodiments, the IACM includes a face tent coupled to a suction tube adapter. The face tent includes a proximal opening configured to be disposed over the mouth and nose of a patient. The face tent further includes a distal opening with a smaller diameter than the proximal opening. A coupler is configured to secure the suction tube adapter to the distal opening of the face tent. The suction tube adapter includes a suction port configured to be coupled to a suction tube for active capture of infectious aerosol and left unconnected for passive capture of infectious aerosol. A viral filter is disposed between the suction port and the face tent to capture infectious aerosol being expelled by the patient (e.g., as a result of exhaled breath, coughing, sneezing, etc.). The IACM further includes one or more one-way valves that are configured to permit airflow into the face tent when the patient is not being provided oxygen through breathing tubes or a nebulizer, or as a redundant measure so that the patient may continue to breath if a medical oxygen supply is somehow cutoff. The one or more one-way valves may also prevent excessive negative pressure from forming within the face tent when suction is being applied through the suction port of the suction tube adapter.

In some embodiments, the one or more one-way valves permit airflow into the face tent indirectly through valve apertures in the suction tube adapter. For example, the suction tube adapter may include a base with a raised central portion extending from the base, where the suction port and the one or more one-way valves are both disposed within the raised central portion of the suction tube adapter. Alternatively, the one or more one-way valves may permit airflow into the face tent directly through valve apertures in the face tent itself. For example, the one or more one-way valves may be disposed within side portions (e.g., left and right sides) of the face tent instead of being disposed within the central portion of the suction tube adapter. In further embodiments, the suction tube adapter and the face tent may each include one or more one-way valves.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is an environmental view of an infectious aerosol capture mask (IACM), in accordance with one or more embodiments of this disclosure.

FIG. 2A is a perspective view of the IACM, in accordance with one or more embodiments of this disclosure.

FIG. 2B is a cross-sectional view of the IACM, in accordance with one or more embodiments of this disclosure.

FIG. 2C is a rear view of the IACM, in accordance with one or more embodiments of this disclosure.

FIG. 3A is a perspective view of a suction tube adapter of the IACM, in accordance with one or more embodiments of this disclosure.

FIG. 3B is a front view of the suction tube adapter of the IACM, in accordance with one or more embodiments of this disclosure.

FIG. 3C is a rear view of the suction tube adapter of the IACM, in accordance with one or more embodiments of this disclosure.

FIG. 4A is a perspective view of a coupler for the suction tube adapter of the IACM, in accordance with one or more embodiments of this disclosure.

FIG. 4B is a front view of the coupler for the suction tube adapter of the IACM, in accordance with one or more embodiments of this disclosure.

FIG. 4C is a rear view of the coupler for the suction tube adapter of the IACM, in accordance with one or more embodiments of this disclosure.

FIG. 5A is a perspective view of a face tent of the IACM, in accordance with one or more embodiments of this disclosure.

FIG. 5B is a front view of the face tent of the IACM, in accordance with one or more embodiments of this disclosure.

FIG. 5C is a rear view of the face tent of the IACM, in accordance with one or more embodiments of this disclosure.

FIG. 6A is a perspective view of the IACM, wherein the face tent includes one or more one-way valves, in accordance with one or more embodiments of this disclosure.

FIG. 6B is an environmental view of the IACM, wherein the face tent includes one or more one-way valves, in accordance with one or more embodiments of this disclosure.

### DETAILED DESCRIPTION

The present disclosure is directed to an infectious aerosol capture mask (IACM) that prevents potential environmental contamination, and therefore potential healthcare worker infection, by effectively capturing aerosolized particles directly from hospital patients. The IACM can be used both actively (with applied suction) and passively (without applied suction) to contain and prevent the spread of infectious aerosol being expelled by a patient (e.g., as a result of exhaled breath, coughing, sneezing, etc.).

As an active system, the IACM incorporates both filtration and vacuum and, as such, can capture the majority if not all exhaled aerosols even if they are being generated at high flow rates. Currently available masks are typically not form, which would be necessary in a passive system to drive all aerosols generated by a nebulizer and/or exhaled by a patient through filters. Thus, with currently available technology, the expiratory gases typically follow the path of least resistance, around the face mask (i.e., the perimeter of the mask where it abuts the patient’s face). This results in leakage of infectious aerosol about the mask perimeter. Consequently, most if not all currently available masks are only effective at low flow rates and cannot support concurrent delivery of high flow oxygen to the patient due to the amount of infectious aerosol leakage that occurs at high flow rates.

The IACM does not suffer from the above-mentioned limitations. Because of its structure and configuration, which are discussed below, the IACM can handle high flow rates when being used actively (i.e., with applied suction). The IACM also gives caregivers the option of using it passively (i.e., without applied suction), further preventing the spread of infectious aerosol by alleviating the need to remove a patient’s mask when the patient is not being connected to oxygen or given a nebulizer treatment. For example, the IACM may be used passively (without applied suction) when a patient is being transported, given emergency medical attention, or during standard procedures that may necessitate the removal of a vacuum or pump.

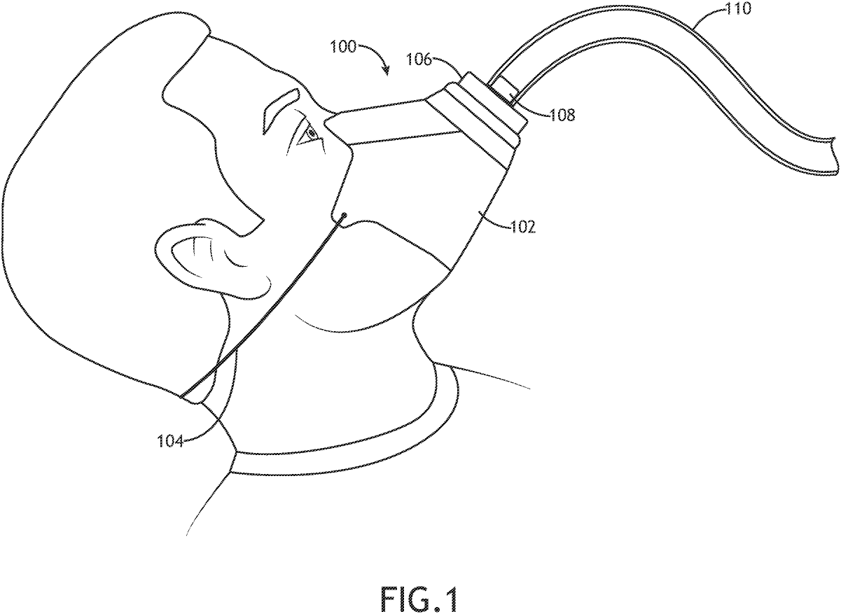

FIG. 1 is an environmental view of an IACM 100. In embodiments, the IACM 100 includes a face tent 102 coupled to a suction tube adapter 106. As shown in FIG. 1, the face tent 102 includes a proximal opening configured to be disposed over the mouth and nose of a patient. The face tent 102 further includes a distal opening with a smaller diameter than the proximal opening. In some embodiments, the face tent 102 is configured to be held tightly against the patient’s face by a head strap 104 (e.g., an elastic head strap, Velcro head strap, tie-on head strap, buckling, buttoning or otherwise fastening head strap, or any combination thereof).

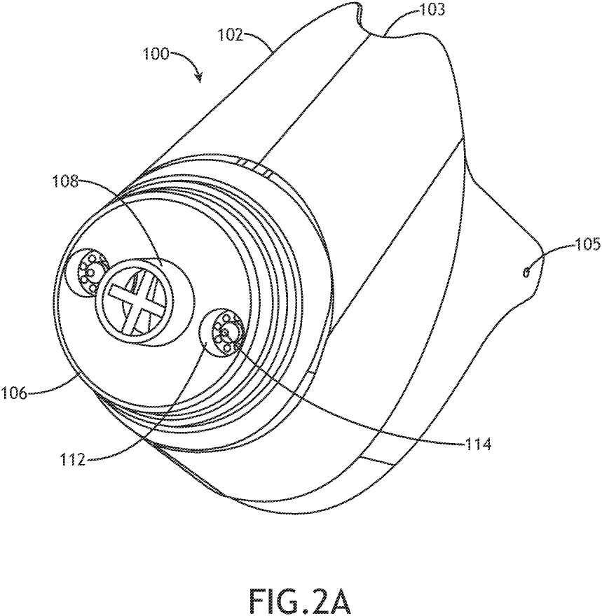

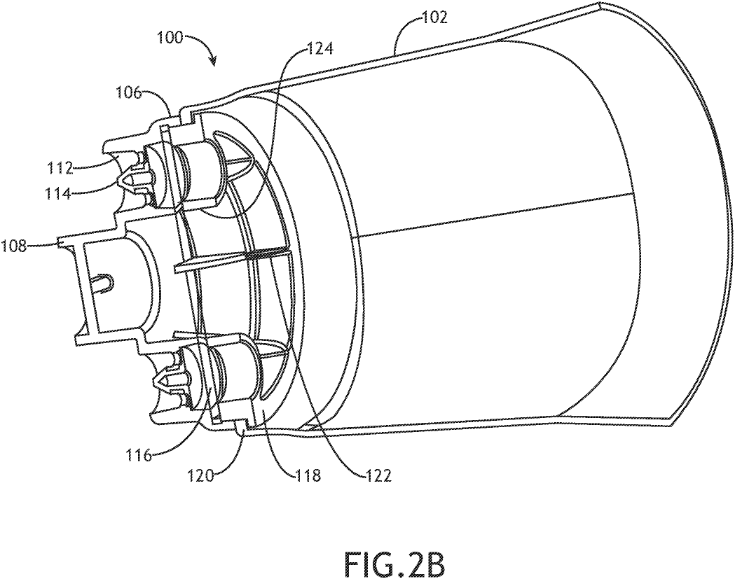

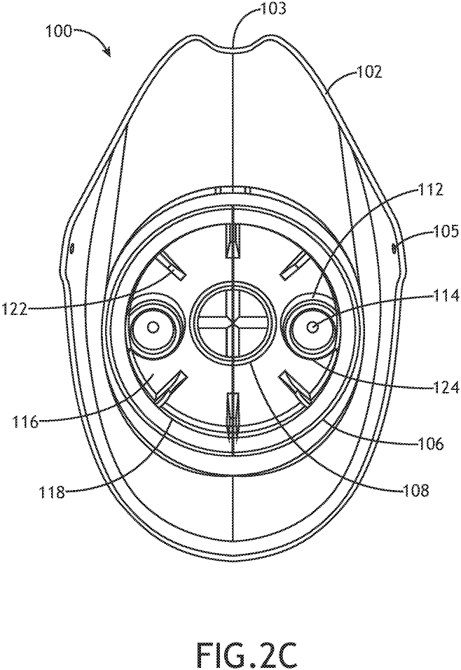

The suction tube adapter 106 includes a suction port 108 configured to be connected to a suction tube 110 for active capture of infectious aerosol and left unconnected for passive capture of infectious aerosol. In embodiments, the suction tube 110 may be coupled to a vacuum source or pump that creates a negative pressure in the suction tube 110 so that fluid (gas and/or liquid) is removed from the IACM 100 through the suction port 108 at a controlled rate. The applied suction force drives aerosols generated by a nebulizer and/or exhaled by a patient through a viral filter 116 (see FIG. 2B) in the suction tube adapter 106.

FIG. 2A is a perspective view of the IACM 100, showing face tent 102 and suction tube adapter 106 structures in more detail.

As shown in FIG. 2A, the face tent 102 may be shaped as an asymmetrical truncated cone with a circular or substantially circular distal opening and a larger, elongated (e.g., elliptical or substantially elliptical) proximal opening. Other geometries may be appropriate. For example, it is contemplated that the face tent 102 may be symmetrical in some embodiments; however, the asymmetrical shape shown in FIG. 2A may better conform the patient’s face. A top portion of the face tent 102 may include an indentation 103 configured to conform to a nasal bridge of the patient. The face tent 102 may further include two tabs configured to extend underneath the patient’s ears, wherein each of the tabs includes a connection point 105 (e.g., an opening, socket, fastener, etc.) for the head strap 104.

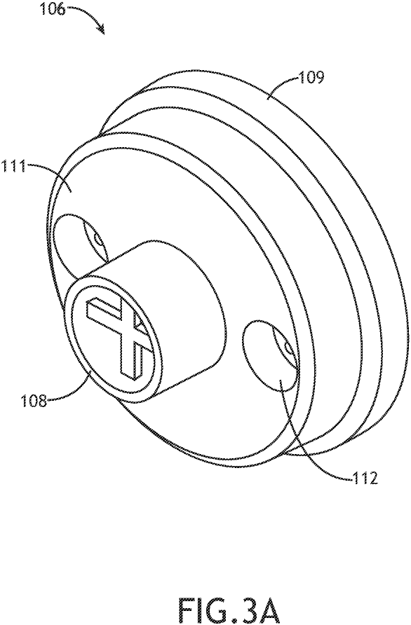

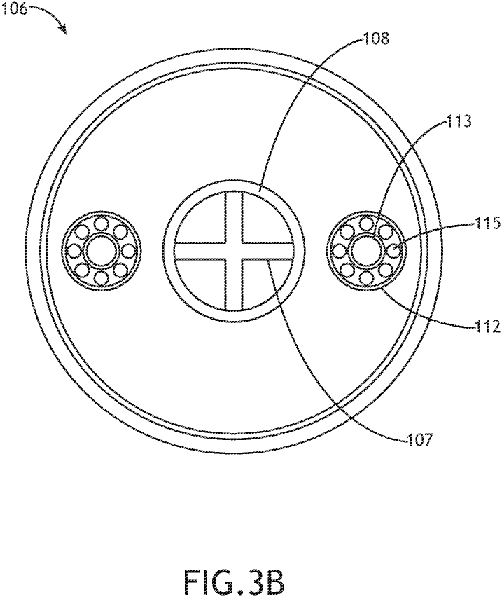

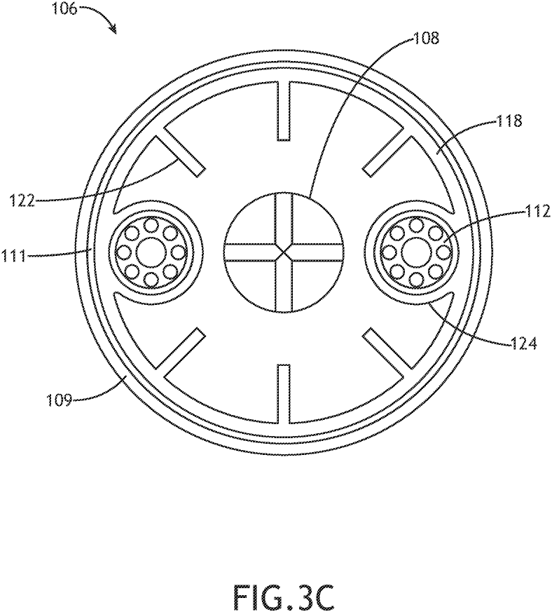

In embodiments, the suction tube adapter 106 includes the suction port 108 at or near the center of the suction tube adapter 106. The suction tube adapter 106 may further include one or more one-way valves 112 adjacent to the suction port 108. For example, in the embodiment illustrated in FIG. 2A, the suction tube adapter 106 includes two one-way valves 112 disposed on either side of the suction port 108. In general, the suction tube adapter 106 may include any number of one-way valves 112 disposed about the suction port 108, but two one-way valves 112 disposed on either side of the suction port 108 may be appropriate for most applications. Each of the one-way valves 112 may include a restrictor 114 that only permits air movement through the one-way valve 112 in a single direction (into the IACM 100) and prevents air movement through the one-way valve 112 in the opposite direction (out of the IACM 100). In some embodiments, the restrictor 114 is a rubber stopper with a flange that covers a plurality of valve apertures 115 (see FIG. 3B) from within the suction tube adapter 106 in order to only permit airflow through the valve apertures 115 into the suction tube adapter 106 and then into the face tent 102.

The one or more one-way valves 112 may be configured to permit airflow through the suction tube adapter 106 and into the face tent 102 when the patient is not being provided oxygen through breathing tubes or a nebulizer, or as a redundant measure so that the patient may continue to breath if a medical oxygen supply is somehow cutoff. The one or more one-way valves 112 may also prevent excessive negative pressure from forming within the face tent 102 when suction is being applied through the suction port 108 of the suction tube adapter 106, thereby preventing any harm to the patient as a result of the applied suction.

FIG. 2B is a cross-sectional view of the IACM 100, showing internal portions of the face tent 102 and the suction tube adapter 106 in more detail.

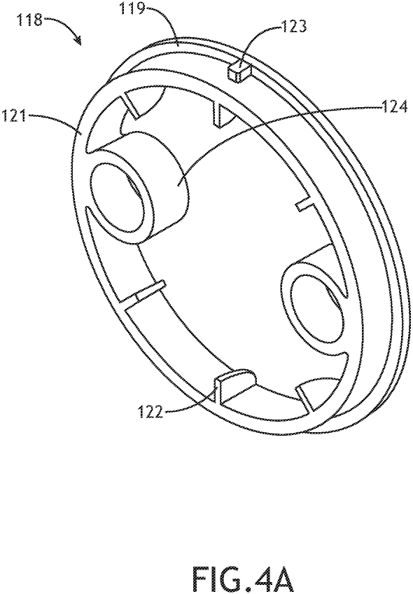

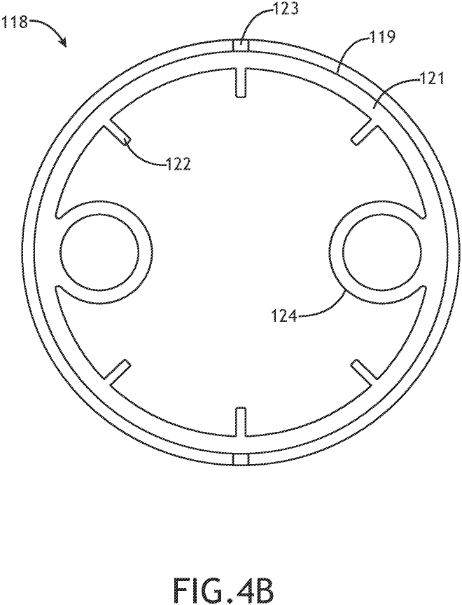

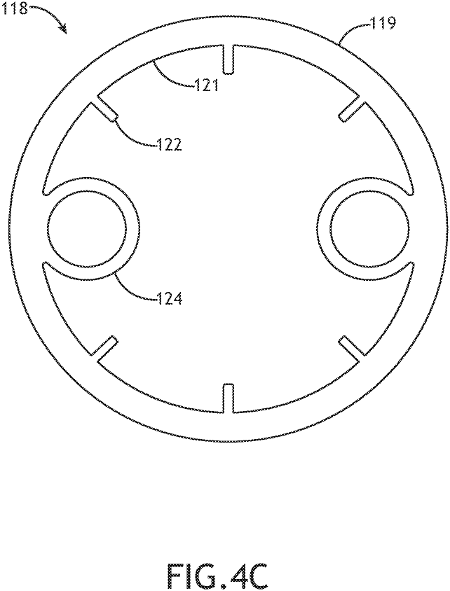

As shown in FIG. 2B, the IACM 100 may include a coupler 118 that is configured to secure the suction tube adapter 106 to the distal opening of the face tent 102. In embodiments, the coupler 118 has an annular frame 121 (see FIG. 4A) and is configured to secure the suction tube adapter 106 to the distal opening of the face tent 102 by holding an inner rim 120 of the distal opening of the face tent 102 within an annular (or otherwise shaped) base 109 (see FIG. 3A) of the suction tube adapter 106. The coupler 118 in the illustrated embodiments is configured to secure the suction tube adapter 106 to the distal opening of the face tent 102 using a snap-fit or interference fit connection between the coupler 118 and the suction tube adapter 106 with the inner rim 120 of the distal opening of the face tent 102 held therebetween. However, in other embodiments, the coupler 118 may be configured to employ other modes of fastening to secure the suction tube adapter 106 to the face tent 102 (e.g., a threaded coupling, latch, lock, or the like).

The viral filter 116 is disposed between the suction port 108 and the face tent 102 to capture infectious aerosol expelled by the patient (e.g., as a result of exhaled breath, coughing, sneezing, etc.), infectious aerosol circulated within the IACM 100 by a nebulizer, or any infectious aerosols that are otherwise present within the face tent 102. As previously noted herein, the suction port 108 may be connected to a suction tube 110 for active capture of infectious aerosol and left unconnected for passive capture of infectious aerosol. In an active use configuration, the suction tube 110 may be coupled to a vacuum source or pump that creates a negative pressure in the suction tube 110 so that fluid (gas and/or liquid) is removed from the IACM 100 through the suction port 108 at a controlled rate. The applied suction force drives aerosols generated by a nebulizer and/or exhaled by the patient through the viral filter 116 so that infectious particles and/or droplets are captured by the viral filter 116 and are therefore prevented from being released into the ambient air.

In embodiments, the coupler 118 is configured to hold the viral filter 116 within the suction tube adapter 106 with the viral filter 116 being pressed firmly against the suction port 108 so that aerosols being drawn out of the IACM 100 through the suction port 108 are not able to circumvent the viral filter 116. In order to firmly secure the viral filter 116 within the suction tube adapter 106 so that it is held against the suction port 108, the coupler 118 may include a plurality of protrusions 122 (e.g., fin-like or tooth-like structures, rods, or alternatively shaped projections) that extend from the annular frame 121 at least part of the way toward a center of the annular frame 121. The protrusions 122 are configured to stabilize the viral filter 116 within the suction tube adapter 106 in between the suction port 108 and the coupler 118 and/or inner rim 120 of the distal opening of the face tent 102. The protrusions 122 can hold the viral filter 116 in place and may also serve to prevent the viral filter 116 from deforming or vibrating when suction is applied as this could undesirably increase the chances of aerosol circumventing the viral filter 116 (e.g., through an air leak from about the perimeter of the viral filter 116).

In some embodiments, the coupler 118 may further include one or more rings 124 extending from the annular frame 121 part of the way toward the center of the annular frame 121. Each of the rings 124 may be configured to be aligned with a corresponding one-way valve 112 in the suction tube adapter 106, so that a portion of the viral filter 116 is disposed between each ring 124 and the one-way valve 112 with which it is aligned. Having a portion of the viral filter 116 disposed over each of the one-way valves 112 helps ensure that, even if some backflow occurs through a one-way valve 112, the viral filter 116 will still be able to prevent the one-way valve 112 from releasing any infectious aerosol out of the IACM 100. The one or more rings 124 also provide an additional safety measure by isolating portions of the viral filter 116 that are used to capture infectious particles/droplets in the aerosols forced through the suction port 108 from the portions of the viral filter 116 that are used to filter the air flow through the one or more one-way valves 112.

FIG. 2C is a rear view of the IACM 100, which further illustrates the structural arrangement and functionality of the face tent 102, suction tube adapter 106, and associated components of the IACM 100.

As shown in FIG. 2C, when the IACM 100 is worn by a patient, the face tent 102 completely covers the patient’s mouth and nose and conforms to the patient’s face to prevent aerosol leakage from about the perimeter of the IACM 100. In passive use, the suction port 108 is left unconnected (i.e., left open to ambient air), but the IACM 100 nevertheless prevents the release of infectious aerosol using the viral filter 116 disposed over the suction port 108 to capture infectious particles/droplets in aerosols exiting the IACM 100. Without applied suction, the performance of the IACM 100 is dependent upon the conformance of the face tent 102 to the patient’s face, but it is contemplated that the IACM 100 may capture at least 60-70% of infectious particles/droplets in aerosols exiting the IACM 100.

When the suction port 108 is connected to the suction tube 110 and suction is applied (i.e., in an active use configuration), the suction force produces one or more flow paths (much like air currents) in the IACM 100 (i.e., within the face tent 102 and suction tube adapter 106) that are directed toward the suction port 108. This drives all fluids (gases, liquids, and/or aerosols) expelled by the patient or circulated within the IACM 100 through the suction port 108, and hence through the viral filter 116. As a result, there is much less chance of any aerosol leakage from about the permitter of the face tent 102 even if the face tent 102 does not fully conform to the patient’s face. Furthermore, when the suction port 108 is connected to the suction tube 110, air can only enter the IACM 100 through the one or more one-way valves 112. As a result, the portions of the viral filter 116 that are disposed over the suction port 108 only experience fluid flow in a single direction (out of the IACM 100), so there is very low chance that any infectious particles/droplets previously have been captured by the viral filter 116 will be subsequently dislodged because incoming air must flow through the one or more one-way valves 112, which may share the viral filter 116 with the suction port 108 in some embodiments but have means (e.g., rings 124) for isolating the respective filter portions used for the one or more one-way valves from the filter portions used for the suction port 108. Without applied suction (active use), the performance of the IACM 100 is much less dependent upon the conformance of the face tent 102 to the patient’s face, and it is contemplated that the IACM 100 may capture at least 80-90% of infectious particles/droplets in aerosols exiting the IACM 100.

FIGS. 3A through 3C illustrate the suction tube adapter 106 in further detail.

As shown in FIG. 3A, the suction tube adapter 106 may include an annular (or otherwise shaped) base 109 with a raised central portion 111 extending from the base 109. The raised central portion 111 may be indented from the outer perimeter of the base 109, where the indentation distinguishes between the raised central portion 111 from the base 109. In embodiments, the base 109 is substantially hollow. As shown in FIG. 3C, this enables the coupler 118 to be disposed within the base 109 so that a snap-fit or interference fit connection can be used to secure the two parts together. As shown in FIG. 2B, the viral filter 116 may be disposed within the base 109. Alternatively, the viral filter 116 may be disposed with raised central portion 111 so long as it is held in place by the coupler 118 when the coupler is used to secure the suction tube adapter 106 to the distal opening of the face tent 102.

In embodiments, the suction port 108 and the one or more one-way valves 112 are both disposed within the raised central portion 111 of the suction tube adapter 106. For example, in the embodiment illustrated in FIGS. 3A through 3C, the suction tube adapter 106 includes two one-way valves 112 disposed on either side of the suction port 108. In general, the suction tube adapter 106 may include any number of one-way valves 112 disposed about the suction port 108, but two one-way valves 112 disposed on either side of the suction port 108 may be appropriate for most applications.

As shown in FIG. 3B, each one-way valve 112 may include a central opening 113 configured to hold a restrictor 114 and a plurality of valve apertures 115 disposed about the central opening 113, wherein the restrictor 114 is a rubber stopper with a flange that covers the valve apertures 115 from within the suction tube adapter 106 (as shown in FIG. 2B) in order to only permit airflow through the valve apertures 115 in a single direction (i.e., into the IACM 100).

In some embodiments, the suction port 108 may include a cross-bar 107 disposed over or within the opening of the suction port 108 to regulate flow through the suction port 108. For example, the cross-bar 107 may prevent air pockets from forming and causing the suction tube 110 to experience a sudden jerk or an otherwise irregular flow rate.

In embodiments, the face tent 102 and/or the suction tube adapter 106 may be formed medical grade plastics. Furthermore, the suction tube adapter 106 may be formed by a common (integrated) injection molded or 3D printed structure that includes the base 109 and the raised central portion 111 with a plurality of openings formed within the raised central portion 111 for the suction port 108 and the one or more one-way valves 112. The face tent 102 may be formed from a flexible plastic (or other substantially nonpermeable material) to enable the face tent 102 to better conform to the patient’s face (e.g., form fitting and conforming to the patient’s cheeks and chin), whereas the suction tube adapter 106 may be formed from a more rigid and/or thicker plastic (or other substantially nonpermeable material).

FIGS. 4A through 4C illustrate the coupler 118 in further detail. As shown in FIG. 4A, the coupler 118 includes an outer rim 119 around the proximal opening of the annular frame 121. The outer rim 119 may further include one or more connectors 123 (e.g., snap-fit or interference fit connectors) for securing the coupler 118 within the base 109 of the suction tube adapter 106 with the inner rim 120 of the distal opening of the face tent 102 held therebetween. For example, in the embodiment illustrated in FIG. 2B, the inner rim 120 of the distal opening of the face tent 102 is held between the outer rim 119 of the proximal opening of the coupler 118 and an inner cavity of the base 109 when the coupler 118 is at least partially disposed within the inner cavity of the base 109. The coupler 118 may include any number of connectors 123. For example, in the embodiment illustrated in FIG. 4B, the coupler 118 includes two connectors 123 disposed on opposite sides of the outer rim 119/annular frame 121. However, in other embodiments, the coupler 118 may include more than two connectors 123 or other types of fasteners (e.g., a threaded fastener, lock, latch, or the like).

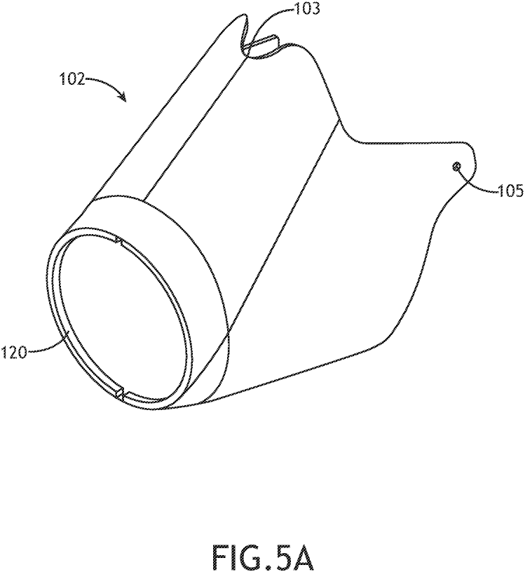

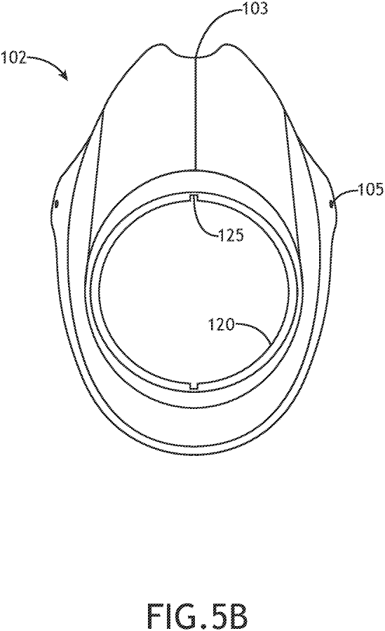

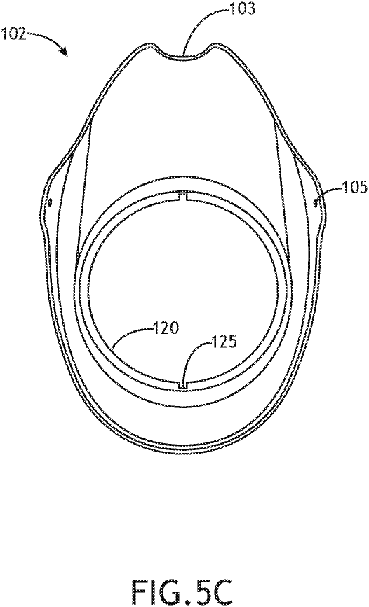

FIGS. 5A through 5C illustrate the face tent 102 in further detail. As discussed above, the face tent 102 includes an inner rim 120 at the distal opening of the face tent 102 to facilitate coupling between the face tent 102 and the suction tube adapter 106. As shown in FIGS. 5B and 5C, in some embodiments, the inner rim 120 includes two or more notches 125 or sockets configured to mate with or let through the connectors 123 of the coupler 118. For example, a process of securing the face tent 102 to the suction tube adapter 106 may include: (1) disposing the coupler 118 within the face tent 102, (2) feeding the connectors 123 into or through the notches 125, and (3) forcing the coupler 118 into the base 109 of the suction tube adapter 106 to form an snap-fit or interference fit connection between the coupler 118 and the suction tube adapter 106 with the inner rim 120 of the distal opening of the face tent 102 held therebetween. The viral filter 116 may be disposed within the suction tube adapter 106 prior the process of securing the face tent 102 to the suction tube adapter 106 in order to achieve the configuration illustrated in FIG. 2B.

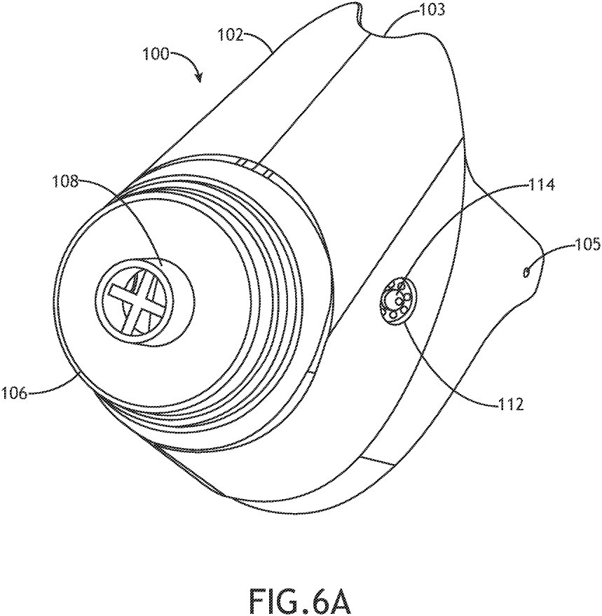

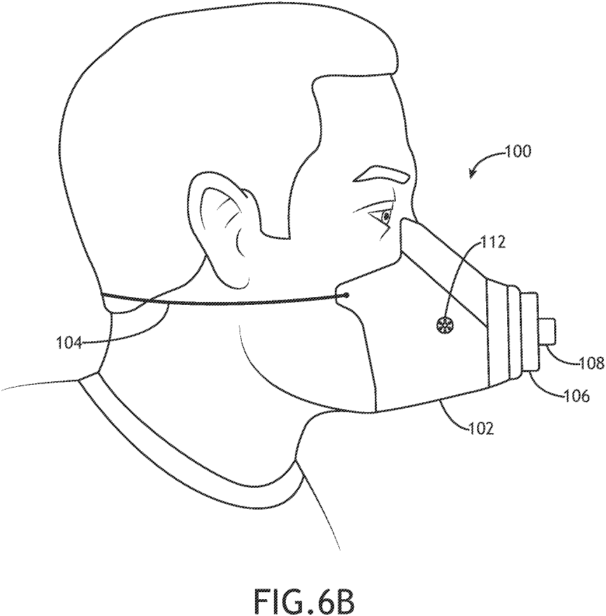

FIGS. 6A and 6B illustrate an alternative embodiment of the IACM 100, where the one or more one-way valves 112 are disposed within side portions (e.g., left and right sides) the face tent 102 instead of being disposed within the suction tube adapter 106. In such embodiments, the suction tube adapter 106 may include the suction port 108 in the raised central portion 111, without any one-way valves 112 disposed in the raised central portion 111. The coupler 118 will typically not include rings 124 in such configurations as there is no need for the rings 124 in such embodiments other than to provide further stability for the viral filter 116. In the embodiment illustrated in FIGS. 6A and 6B, the one-way valves 112 may be similarly structured to the one-way valves 112 discussed with reference to FIGS. 1 through 5C, except that the one-way valves 112 are now disposed in the face tent 102 itself. In this regard, the face tent 102 may include a central opening surrounded by valve apertures (much like the central opening 113 and valve apertures 115 illustrated in FIG. 3B), where the restrictor 114 may be a rubber stopper with a flange that covers the valve apertures from within the face tent 102 in order to only permit airflow through the valve apertures in a single direction (i.e., into the face tent 102/IACM 100).

In further embodiments, the suction tube adapter 106 and the face tent 102 may each include one or more one-way valves 112. For example, the suction tube adapter 106 as illustrated in FIGS. 1A through 5C may be combined with the face tent 102 as illustrated in FIGS. 6A and 6B.

The IACM 100, in any of the configurations described above, may further include an opening or port in the face tent 102 for a nebulizer, breathing tube, or any other medical fluid (gas and/or liquid) supply line to be provided for the patient. For example, a nebulizer, breathing tube, or any other medical fluid (gas and/or liquid) supply line may be fed through an opening or coupled to a port above or below the suction tube adapter 106. In some embodiments, the face tent 102 itself may include a portion of nebulizer integrated within the structure of the face tent 102. For example, the face tent 102 may include a nebulizer mouthpiece extending into the face tent 102 as part of its overall structure (e.g., formed from the same injection mold or 3D print). Alternatively, the suction tube adapter 106 may include an opening or port for a nebulizer, breathing tube, or any other medical fluid (gas and/or liquid) supply line to be provided for the patient. For example, a nebulizer, breathing tube, or any other medical fluid (gas and/or liquid) supply line may be fed through an opening or coupled to a port in the suction tube adapter 106. In some embodiments, the suction tube adapter 106 itself may include a portion of nebulizer integrated within the structure of the suction tube adapter 106. For example, the suction tube adapter 106 may include a nebulizer mouthpiece extending into the suction tube adapter 106 as part of its overall structure (e.g., formed from the same injection mold or 3D print). It is further contemplated that, in some embodiments, one or more one-way valves 112 in the face tent 102 or the suction tube adapter 106 may be reconfigured to connect a nebulizer, breathing tube, or any other medical fluid (gas and/or liquid) supply line through the IACM 100.

In embodiments, the IACM 100 may further include a suction indicator (e.g., a light-emitting diode (LED) or other visual indicator, audible indicator, or signal transmitter) that is configured to indicate when suction is applied based on a flow switch embedded within or coupled to the suction port 108. For example, the suction port 108 may include or may be coupled to an electromechanical or electromagnetic flow switch that is configured to activate the suction indicator when active suction (e.g., fluid flow above a predefined threshold) is detected within the suction port 108 or alternatively within the suction tube 110.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

What is claimed is:

1. An infectious aerosol capture mask, comprising:
a face tent configured to cover a mouth and nose of a patient, the face tent including a proximal opening configured to be disposed over the mouth and nose of the patient, the face tent further including a distal opening with a smaller diameter than the proximal opening;
a suction tube adapter including a suction port configured to be coupled to a suction tube for active capture of infectious aerosol and left unconnected for passive capture of infectious aerosol;
a coupler configured to secure the suction tube adapter to the distal opening of the face tent;
a viral filter disposed between the suction port and the face tent; and
one or more one-way valves configured to permit airflow into the face tent.

2. The infectious aerosol capture mask of claim 1, wherein the suction port is in a central region of the suction tube adapter.

3. The infectious aerosol capture mask of claim 2, wherein the one or more one-way valves are disposed about the suction port in the central region of the suction tube adapter.

4. The infectious aerosol capture mask of claim 1, wherein the suction tube adapter comprises a common injection molded or 3D printed structure with a base and a raised central portion with a plurality of openings formed within the raised central portion for the suction port and the one or more one-way valves.

5. The infectious aerosol capture mask of claim 1, wherein the coupler comprises an annular frame and is configured to secure the suction tube adapter to the distal opening of the face tent by holding an inner rim of the distal opening of the face tent within a base of the suction tube adapter.

6. The infectious aerosol capture mask of claim 5, wherein the coupler further comprises a plurality of protrusions extending toward a center of the annular frame, wherein the plurality of protrusions are configured to hold the viral filter within the suction tube adapter in between the suction port and the inner rim of the distal opening of the face tent.

7. The infectious aerosol capture mask of claim 5, wherein coupler further comprises one or more rings extending toward a center of the annular frame, wherein the one or more rings are configured to be aligned with the one or more one-way valves, wherein a portion of the viral filter is disposed between the one or more one-way valves and the one or more rings.

8. The infectious aerosol capture mask of claim 1, wherein each of the one or more one-way valves includes a rubber stopper with a flange that covers a plurality of valve apertures from within the suction tube adapter in order to only permit airflow through the plurality of valve apertures into the suction tube adapter and then into the face tent.

9. The infectious aerosol capture mask of claim 1, wherein the face tent includes the one or more valves.

10. The infectious aerosol capture mask of claim 9, wherein each of the one or more one-way valves includes a rubber stopper with a flange that covers a plurality of valve apertures from within the face tent in order to only permit airflow through the plurality of valve apertures into the face tent.

11. A suction tube adapter, comprising:
a base;
a raised central portion extending from the base;
a suction port in the raised central portion, the suction port configured to be coupled to a suction tube for active capture of infectious aerosol and left unconnected for passive capture of infectious aerosol;
one or more one-way valves in the raised central portion, the one or more one-way valves configured to permit airflow into a face tent through the suction tube adapter;
a coupler configured to secure the base to a distal opening of the face tent; and
a viral filter disposed between the suction port and the coupler.

12. The suction tube adapter of claim 11, wherein the suction port is in a central region of the raised central portion, and the one or more one-way valves are disposed about the suction port in the central region of the raised central portion.

13. The suction tube adapter of claim 11, wherein the base and the raised central portion with a plurality of openings formed within the raised central portion for the suction port and the one or more one-way valves are all part of a common injection molded or 3D printed structure.

14. The suction tube adapter of claim 11, wherein the coupler comprises an annular frame and is configured to secure the base to the distal opening of the face tent by holding an inner rim of the distal opening of the face tent within the base.

15. The suction tube adapter of claim 14, wherein the coupler further comprises a plurality of protrusions extending toward a center of the annular frame, wherein the plurality of protrusions are configured to hold the viral filter in between the suction port and the inner rim of the distal opening of the face tent.

16. The suction tube adapter of claim 14, wherein coupler further comprises one or more rings extending toward a center of the annular frame, wherein the one or more rings are configured to be aligned with the one or more one-way valves, wherein a portion of the viral filter is disposed between the one or more one-way valves and the one or more rings.

17. The suction tube adapter of claim 11, wherein each of the one or more one-way valves includes a rubber stopper with a flange that covers a plurality of valve apertures from within the central raised portion in order to only permit airflow through the plurality of valve apertures into the suction tube adapter and then into the face tent.

18. A suction tube adapter, comprising:
a base;
a raised central portion extending from the base;
a suction port in the raised central portion, the suction port configured to be coupled to a suction tube for active capture of infectious aerosol and left unconnected for passive capture of infectious aerosol;
a coupler configured to secure the base to a distal opening of a face tent; and
a viral filter disposed between the suction port and the coupler.

19. The suction tube adapter of claim 18, wherein the coupler comprises an annular frame and is configured to secure the base to the distal opening of the face tent by holding an inner rim of the distal opening of the face tent within the base.

20. The suction tube adapter of claim 19, wherein the coupler further comprises a plurality of protrusions extending toward a center of the annular frame, wherein the plurality of protrusions are configured to hold the viral filter in between the suction port and the inner rim of the distal opening of the face tent.
